(19) United States
(12) Patent Application Publication
(10) Pub. No.: US 2025/0179124 A1
(43) Pub. Date: Jun. 5, 2025

(54) BETA-CATENIN PROTEIN DEGRADATION

(71) Applicant: UbiquiTx, New York, NY (US)

(72) Inventors: Pranam CHATTERJEE, New York, NY (US); Suhaas BHAT, New York, NY (US); Kalyan PALEPU, New York, NY (US); Matthew DELISA, Ithaca, NY (US); Tina YE, Ithaca, NY (US)

(73) Assignee: UbiquiTx, New York, NY (US)

(21) Appl. No.: 18/836,533

(22) PCT Filed: Feb. 7, 2023
(86) PCT No.: PCT/US2023/062124

§ 371 Date: Aug. 7, 2024

Related U.S. Application Data
(60) Provisional application No. 63/307,606, filed on Feb. 7, 2022.

(51) Int. Cl. C07K 14/00; A61K 38/00; C12N 9/10

(52) U.S. Cl. CPC C07K 14/001; C12N 9/104; A61K 38/00; C07K 2319/70

(57) ABSTRACT

An isolated chimeric molecule is provided comprising: (i) a degradation domain comprising an E3 ubiquitin ligase motif without lysine residues; (ii) a targeting domain comprising a substrate-binding motif which is heterologous to the E3 ubiquitin ligase motif and configured to bind to Beta-Catenin; and (iii) a linker coupling said degradation domain to said targeting domain.

### CROSS-REFERENCE TO RELATED APPLICATION

The present application claims the benefit of priority to U.S. provisional patent Application No. 63/307,606 filed on Feb. 7, 2022, which is incorporated herein by reference in its entirety.

### SEQUENCE LISTING

The instant application contains a Sequence Listing which has been filed electronically in XML format and is hereby incorporated by reference in its entirety. Said XML copy, created on Feb. 4, 2023 is named 05286_010968-WO0_SL and is 25.5 bytes in size.

### FIELD OF INVENTION

The present disclosure relates to systems and methods for generating minimal, specific, nucleotide encodable, protein to proteasome linkers for use in diagnostic, analytic and therapeutic applications and compositions relating to the same.

### BACKGROUND OF THE INVENTION

Targeted protein depletion is a key method of disrupting protein-protein interactions and protein function in vivo. Protein synthesis can be blocked at various levels. At the DNA level, protein coding genes can be disrupted using genome editing tools, such as zinc-finger nucleases, TALENs, and CRISPR-Cas9. At the post-transcriptional level, methods such as RNAi or CRISPR-Cas13 can be used for degrading targeted messenger RNAs (mRNAs). Finally, at the translational level, antisense oligonucleotides can be utilized to hybridize to the mRNA and block the progression of the translation initiation complex from the 5′ cap to the start codon.

The most rapid and acute method of protein degradation intracellularly, however, is at the post-translational level. Specifically, E3 ubiquitin ligases can tag endogenous proteins for subsequent degradation in the proteasome. Thus, by guiding E3 ubiquitin ligases to a protein of interest, one can mediate its depletion. Numerous previous works have attempted to redirect E3 ubiquitin ligases by replacing their natural protein binding domains with those targeting specific proteins. For example, U.S. Pat. No. 11,192,492, herein incorporated by reference in its entirety, discloses the use of E3 ubiquitin ligases in connection with the protein depletion.

Regulatory T cells (Tregs) are a specialized subpopulation of T cells that act to suppress immune response, thereby maintaining homeostasis and self-tolerance. Conversely, the immunosuppressive activity of Tregs may contribute to the progression of cancer or infectious diseases by preventing the induction of specific immune responses.

Beta-catenin (β-catenin) is a multifunctional, 90 kD protein that contributes to cell development under normal physiological conditions. β-Catenin is a crucial transcriptional factor in Wingless-Int (Wnt) signaling and plays important role in stem cell renewal and organ regeneration. Imbalance in the structural and signaling properties of β-catenin often results in disease and deregulated growth connected to cancer and metastasis. The E3 ubiquitin ligase TrCP1 (also known as β-TrCP) can recognize β-catenin as its substrate through a short linear motif on the disordered N-terminus. Beta-catenin loss-of-function mutations will cause β-catenin to translocate to the nucleus without any external stimulus and continuously drive transcription of its target genes. Increased nuclear β-catenin levels have been noted in basal cell carcinoma (BCC), head and neck squamous cell carcinoma (HNSCC), prostate cancer (CaP), pilomatrixoma (PTR) and medulloblastoma (MDB). Thus, Beta-catenin serves as a druggable target for therapeutic modalities in the tumor microenvironment.

Attempts have been made to computationally determine guide proteins that when linked to E3 ubiquitin ligase would allow for targeted degradation of an intended target substrate. For example, the following references, which are herein incorporated by reference as if presented in their respective entireties, describe approaches to the development of specific targeted degraders: U.S. Provisional Application No. 63/423,320, entitled: “Sequence-Based Framework to Design Peptide-Guided Degraders.” U.S. Provisional Application No. 63/344,820, entitled: “Contrastive Learning for Peptide Based Degrader Design and Uses Thereof.”, U.S. Provisional Application No. 63/032,513, entitled: “Minimal Peptide Fusions for Targeted Intracellular Degradation.” and U.S. Patent Application US20140112922A1, entitled: “Targeted protein silencing using chimeras between antibodies and ubiquitination enzymes.”

Furthermore, the following references are also incorporated by reference as if presented in their respective entireties: Gaj, et al. ZFN, TALEN, and CRISPR/Cas-based methods for genome engineering. Trends Biotechnol. (2013) Boettcher, et al. Choosing the right tool for the job: RNAi, TALEN or CRISPR. Mol. Cell (2015). Eisen, J. S. et al. Controlling morpholino experiments: don't stop making antisense. Development (2008). Ardley, et al. E3 ubiquitin ligases. Essays Biochem. (2005). Gosink, M. M. et al. Redirecting the specificity of ubiquitination by modifying ubiquitin-conjugating enzymes. PNAS (1995). Zhou, et al. Harnessing the ubiquitination machinery to target the degradation of specific cellular proteins. Mol. Cell (2000). Su, et al. Eradication of pathogenic β-catenin by Skp1/Cullin/F box ubiquitination machinery. PNAS (2003). Kwon, et al. Different molecular complexes that mediate transcriptional induction and repression by FoxP3. Nat. Immunol. (2018). Casares, et al. A Peptide Inhibitor of FOXP3 Impairs Regulatory T Cell Activity and Improves Vaccine Efficacy in Mice. J Immunol. (2010). Rudensky. Regulatory T Cells and Foxp3. Immunol Rev. (2012). Sedan, et al. Peptiderive server: derive peptide inhibitors from protein-protein interactions. Nucleic Acids Research (2016). Raveh, et al. Rosetta FlexPepDock ab-initio: Simultaneous Folding, Docking and Refinement of Peptides onto Their Receptors. PLOS ONE (2011). Portnoff, et al. Ubiquibodies, Synthetic E3 Ubiquitin Ligases Endowed with Unnatural Substrate Specificity for Targeted Protein Silencing. J Biol Chem. (2014). Lozano, et al. Blockage of FOXP3 transcription factor dimerization and FOXP3/AML1 interaction inhibits T regulatory cell activity: sequence optimization of a peptide inhibitor. Oncotarget (2017).

### SUMMARY OF THE INVENTION

In one or more particular implementations or embodiments of the invention described, one or more peptide-E3 ubiquitin ligase fusions are provided that allow for the targeted degradation of endogenous, cytosolic β-catenin. Without reference to any specific theory, the inventors have found that structure agnostic large language models can be developed and trained to generate peptides that have properties allowing for the binding of endogenous expressed Beta-catenin proteins and degradation by E3 ubiquitin ligase.

In one or more implementations of the present invention, a computer implemented method is provided for generating a protein to proteasome linker using a sequence based machine learning model. In a further implementation, the machine learning model is further configured to generate a plurality of potential amino acid sequences that can link or bind to Beta-Catenin and be fused to a E3 ubiquitin ligase. For example, one or more computationally derived sequences are provided that bind the target protein and induce their degradation when fused to modular E3 ubiquitin ligase domains.

In yet a further implementation, an isolated chimeric molecule is provided comprising: (i) a degradation domain comprising an E3 ubiquitin ligase motif without lysine residues; (ii) a targeting domain comprising a substrate-binding motif which is heterologous to the E3 ubiquitin ligase motif and configured to bind to Beta-Catenin; and (iii) a linker coupling said degradation domain to said targeting domain.

In the further implementation, the targeting domain has a length of less than 30 amino acids.

In a further implementation of the subject matter described herein, a minimal, specific, nucleotide encodable, protein to proteasome linker is provided. In a particular implementation or embodiment, the linker of described includes a peptide-E3 ubiquitin ligase fusion in which said peptide binds to Beta-Catenin, of amino acid sequence:

(SEQ ID NO.: 26)
MATQADLMELDMAMEPDRKAAVSHWQQQSYLDSGIHSGATTTAPSLSGK
GNPEEEDVDTSQVLYEWEQGFSQSFTQEQVADIDGQYAMTRAQRVRAAM
FPETLDEGMQIPSTQFDAAHPTNVQRLAEPSQMLKHAVVNLINYQDDAE
LATRAIPELTKLLNDEDQVVVNKAAVMVHQLSKKEASRHAIMRSPQMVS
AIVRTMQNTNDVETARCTAGTLHNLSHHREGLLAIFKSGGIPALVKMLG
SPVDSVLFYAITTLHNLLLHQEGAKMAVRLAGGLQKMVALLNKTNVKFL
AITTDCLQILAYGNQESKLIILASGGPQALVNIMRTYTYEKLLWTTSRV
LKVLSVCSSNKPAIVEAGGMQALGLHLTDPSQRLVQNCLWTLRNLSDAA
TKQEGMEGLLGTLVQLLGSDDINVVTCAAGILSNLTCNNYKNKMMVCQV
GGIEALVRTVLRAGDREDITEPAICALRHLTSRHQEAEMAQNAVRLHYG
LPVVVKLLHPPSHWPLIKATVGLIRNLALCPANHAPLREQGAIPRLVQL
LVRAHQDTQRRTSMGGTQQQFVEGVRMEEIVEGCTGALHILARDVHNRI
VIRGLNTIPLFVQLLYSPIENIQRVAAGVLCELAQDKEAAEAIEAEGAT
APLTELLHSRNEGVATYAAAVLFRMSEDKPQDYKKRLSVELTSSLFRTE
PMAWNETADLGLDIGAQGEPLGYRQDDPSYRSFHSGGYGQDALGMDPMM
EHEMGGHHPGADYPVDGLPDLGHAQDLMDGLPPGDSNQLAWFDTDL

In a further implementation, the linker described includes a peptide-E3 ubiquitin ligase fusion in which said peptide is any of the amino acid sequence SEQ ID No.: 1-SEQ ID NO.: 25 and SEQ ID No.: 28-33.

In yet a further implementation, a peptide-E3 ubiquitin ligase fusion is provided in which said E3 ubiquitin ligase is of amino acid sequence:

(SEQ ID NO.: 27)
RLNFGDDIPSALRIAKKKRWNSIEERRIHQESELHSYLSRLIAAERERE
LEECQRNHEGDEDDSHVRAQQACIEAKHDKYMADMDELFSQVDEKRKKR
DIPDYLCGKISFELMREPCITPSGITYDRKDIEEHLQRVGHFDPVTRSP
LTQEQLIPNLAMKEVIDAFISENGWVEDY.

In yet a further implementation, a peptide-E3 ubiquitin ligase fusion is provided where the peptide is a sequence possessing sequence homology of greater than 80% to any of the amino acid sequences SEQ ID No.: 1-SEQ ID NO.: 24 and SEQ ID No.: 28-35.

In yet a further implementation, a peptide-based therapeutic is provided comprising the polynucleotide of any of the preceding implementations coupled a delivery vector in which said delivery vector may be either a virus or micelle.

In yet a further implementation, a peptide-based therapeutic is provided comprising the fusions of any of the preceding polynucleotide sequences in which said peptide fusion is further fused to a cell penetrating motif or a cell surface receptor binding motif.

In yet a further implementation, a peptide-based therapeutic is provided wherein the peptide-E3 ubiquitin ligase is provided within a lipid nano-particle (LNP) or an adeno-associated vectors.

### BRIEF DESCRIPTION OF THE DRAWINGS

FIG. 1 illustrates degradation of endogenous β-catenin in the cytosolic fraction of DLD1 cells.

FIG. 2 illustrates a luciferase reporter assay of β-catenin/TCF transcriptional activity.

FIG. 3 illustrates β-catenin binding activity for computationally derived uAbs.

FIG. 4 illustrates a volcano plot of differentially abundant proteins.

FIG. 5 illustrates the abundance of β-catenin in the presence and absence of the computationally derived uAbs.

FIG. 6 illustrates a flow diagram for the generation of specific guide peptides.

### DETAILED DESCRIPTION OF THE INVENTION

As used herein and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

As used herein and as well understood in the art, the term an “effective amount,” “sufficient amount” or “therapeutically effective amount” of an agent as used herein interchangeably, is that amount sufficient to effectuate beneficial or desired results, including preclinical and/or clinical results and, as such, an “effective amount” or its variants depends upon the context in which it is being applied. The response is in some embodiments preventative, in others therapeutic, and in others a combination thereof. The term “effective amount” also includes the amount of a compound of the disclosure, which is “therapeutically effective” and which avoids or substantially attenuates undesirable side effects.

As used herein and as well known in the art, and unless otherwise defined, the term “subject” means an animal, including but not limited a human, monkey, cow, horse, sheep, pig, chicken, turkey, quail, cat, dog, mouse, rat, rabbit, or guinea pig. In one embodiment, the subject is a mammal and in another embodiment the subject is a human patient.

As used herein, the term “homologous” refers to the subunit sequence similarity between two polymeric molecules, e.g., between two nucleic acid molecules, such as two DNA molecules or two RNA molecules, or between two protein molecules. When a subunit position in both of the two molecules is occupied by the same monomeric subunit; e.g., if a position in each of two DNA molecules is occupied by adenine, they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′-ATTGCC-5′ and 3′-TATGGC-5′ are 50% homologous. As used herein, “homology” is used synonymously with “identity.”

As used herein, the term “substantially the same” amino acid sequence is defined as a sequence with at least 70%, preferably at least about 80%, more preferably at least about 90%, even more preferably at least about 95%, and most preferably at least 99% homology to another amino acid sequence, as determined by the FASTA search method in accordance with Pearson & Lipman, Proc. Natl. Inst. Acad. Sci. USA 1988, 85:2444-2448.

The present disclosure relates to systems and methods for generating, using a peptide generation model, configured as code executing on a computer, one or more minimal, specific, nucleotide encodable, protein to proteasome linkers for use in diagnostic, analytic and therapeutic applications and compositions relating to the same. Using such a peptide generation model, peptides can be experimentally generated that induce robust degradation of diverse pathogenic targets in human cells.

In a particular implementation, an isolated chimeric molecule is provided where the chimeric molecules comprises: (i) a degradation domain comprising an E3 ubiquitin ligase motif without lysine residues; (ii) a targeting domain comprising a Beta-Catenin binding motif which is heterologous to the E3 ubiquitin ligase motif; and (iii) a linker coupling said degradation domain to said targeting domain.

As described, protein binders, including FN3s, DARPins, nanobodies, and peptides can be bound to various E3 ubiquitin ligase domains, to enable binding, selective ubiquitination, and intracellular degradation of diverse pathogenic targets of interest. Such combination of protein binders and E3 ubiquitin ligase domains are referred to herein as uAbs or ubiquibodies.

As described herein, one or more computer implemented methods are used to generate a peptide or protein guide that will bind to the intended biological target of interest. As used herein, the this guide or binder peptide or protein is configured to bind to a biological target of interest as well form a fusion with a regulatory protein, such as but not limited to, E3 ubiquitin ligase.

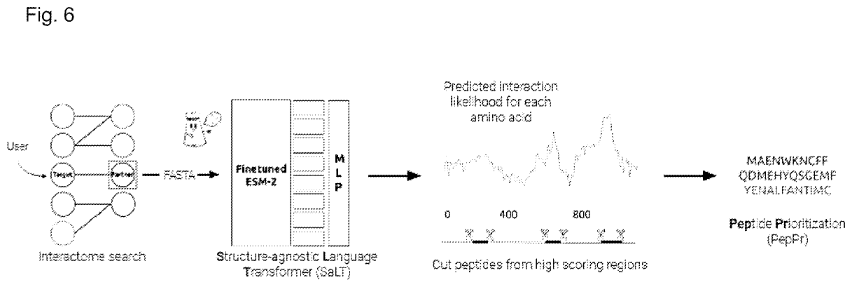

In order to generate a uAb complex for targeted degradation of a biological target, a binder or guide peptide must be generated. As shown in FIG. 6, a target (such as beta-catenin) is provided to a peptide generation system. Here the system is configured to take as an input an amino acid sequence and generate a guide or binder peptide for use in the degradation of the supplied target.

For example, a structure agnostic model, such as a trained large language model, neural network, support vector machine, or a combination thereof, is used to generate a model of amino acid binding probabilities. Based on these probabilities, short (less than 30 AA) peptides are generated that are predicted to have strong binding affinity with the target. It will be appreciated that the structure agnostic language models described herein, are provided in more detail in Brixi, G., Ye, T., et al. Design of Peptide-Guided Protein Degraders with Structure-Agnostic Language Models. Nat Biotechnol (2022), herein incorporated by reference in its entirety. Using such structure agnostic models, herein referred to as Peptide Generators, a sequence can be generated that is intended to bind to the biological target as well as form a peptide-E3 ubiquitin ligase fusion.

As further described herein, the Peptide Generators are configured to reliably and efficiently generate peptides that, when experimentally integrated within a uAb construct, induce robust degradation of diverse pathogenic targets in human cells.

By way of one particular example, the Peptide Generators are implemented in one or more accessible computer platforms or services. For instance, one or more computers are configured to receive a biological target in the form of an amino acid sequence corresponding to Beta-catenin. Here, one or more Peptide Generators was configured to perform one or more high-throughput in silico truncations to the Beta-catenin heterodimer. For example, the Peptide Generators used Peptiderive (of Rosetta Commons) and FlexPepDock (of Hebrew University of Jerusalem) servers to conduct such analysis. This approach minimized the Beta-catenin protein structure to the sufficient components needed for binding to the alternate chain of Beta-catenin. A Peptidrive algorithm was applied multiple times for each peptide length between 10 and 23 amino acids to find candidates derived from each Beta-catenin amino acid chain which binds to the paired chain with high affinity. Each candidate protein was computationally relaxed, and those with the lowest total energy score, and thus highest binding affinity, were selected for experimental analysis.

Candidate peptide sequences were fused the via a short, flexible linker of GSGSG to the 5′ end of CHIPΔTPR, an optimized human-derived E3 ubiquitin ligase, as described by Portnoff, et al. Similar fusions with Beta-catenin-targeting peptides identified previously through experimental screening assays were conducted.

The inventors have found that a subset of the evaluated peptide fusions (shown in Table 1) are capable of mediating robust degradation of Beta-catenin-sfGFP (superfolder green fluorescent protein) fusion proteins within HEK239T cells.

TABLE 1

| | | | | | Relative | |
|---|---|---|---|---|---|---|
| | | Peptide | | Interface | interface | |
| Receptor | Partner | length | Position | score | score (%) | Sequence |
| C | A | 10 | 245 | −3.909 | 15.95 | GHRAAVNVVD |
| C | A | 15 | 331 | −4.847 | 19.77 | RCIRFDNKRIVSGAY |
| C | A | 17 | 329 | −6.045 | 24.65 | LVRCIRFDNKRIVSGAY |
| C | A | 18 | 248 | −10.455 | 28.78 | AAVNVVDFDDKYIVSASG |
| C | A | 19 | 247 | −13.007 | 35.8 | RAAVNVVDFDDKYIVSASG |
| C | A | 20 | 246 | −13.128 | 36.14 | HRAAVNVVDFDDKYIVSASG |
| C | A | 10 | 246 | −8.537 | 22.87 | HRAAVNVVDF |
| C | A | 17 | 249 | −9.888 | 26.48 | AVNVVDFDDKYIVSASG |
| C | A | 10 | 143 | −12.88 | 22.91 | TKHKWEAAHV |
| C | A | 12 | 62 | −13 | 23.12 | GETRKVKAHSQT |
| C | A | 15 | 63 | −18.211 | 32.39 | ETRKVKAHSQTHRVD |
| C | A | 16 | 62 | −18.347 | 32.63 | GETRKVKAHSQTHRVD |
| C | A | 18 | 63 | −19.277 | 34.28 | ETRKVKAHSQTHRVDLGT |
| C | A | 19 | 63 | −20.551 | 36.55 | ETRKVKAHSQTHRVDLGTL |
| C | A | 20 | 62 | −20.687 | 36.79 | GETRKVKAHSQTHRVDLGTL |
| C | A | 22 | 63 | −23.863 | 42.44 | ETRKVKAHSQTHRVDLGTLRGY |
| C | A | 23 | 62 | −23.999 | 42.68 | GETRKVKAHSQTHRVDLGTLRGY |
| C | A | 13 | 143 | −15.463 | 23.08 | TKHKWEAAHVAEQ |
| C | A | 14 | 146 | −17.066 | 25.47 | KWEAAHVAEQLRAY |
| C | A | 17 | 143 | −21.855 | 32.61 | TKHKWEAAHVAEQLRAY |
| C | A | 21 | 143 | −23.112 | 34.49 | TKHKWEAAHVAEQLRAYLEGT |
| C | A | 22 | 146 | −23.451 | 36.85 | KWEAAHVAEQLRAYLEGTCVEW |
| C | A | 10 | 323 | −9.196 | 35.68 | HEELVRCIRF |
| C | A | 10 | 324 | −9.458 | 28.04 | EELVRCIRFD |
| C | A | 16 | 327 | −11.317 | 33.56 | VRCIRFDNKRIVSGAY |

In one or more alternative implementations, the Peptide Generator is based upon Meta AI's ESM-2 model (https://github.com/facebookresearch/esm) with a neural network head trained to classify the per amino acid interacting positions. In a particular configuration, the final three layers of ESM-2 650M were fine tuned together with a four layer fully connected neural network classification head which processes each position output of ESM-2 to predict a per position probability. Here, each protein is passed to the model with the per amino acid binary class as the target for cross entropy loss: −(ylog(p)+(1−y)log(1−p)) where y is the binary class label (0=nonbinding and 1=binding) and p is the predicted probability of the amino acid belonging to a binding site.

In yet a further implementation, predictions made by the Peptide Generator were converted to peptides by adding contiguous amino acids from positions with high average predicted scores and sampling non-overlapping motifs. After candidate peptide derivation, DNA plasmids expressing eight peptides of variable lengths (≤18 amino acids) for each target were experimentally cloned for each method. These peptides were directly fused to the CHIPΔTPR ubiquitination domain via a short glycine-serine linker (GSGSG). These vectors were co-transfected into human HEK293T cells alongside plasmids expressing the target protein fused to superfolder green fluorescent protein (sfGFP). As part of the analysis of the efficacy of the peptides to form uAbs for targeted β-catenin modulation, the reduction of sfGFP signal (and thus target degradation) via flow cytometry versus a uAb control involving a non-targeting, scrambled control peptide was evaluated.

### Linker Sequence

In one or more further implementations, the Peptide Generator using or incorporating structural information is configured to generate a peptide having a relatively short sequence, less than 50 amino acids (AA), and a relatively high binding affinity for the proposed target. For example, the Peptide Generator is configured to provide the peptide of a peptide-E3 ubiquitin ligase fusion in which said peptide is one of the following of amino acid sequences:

SEQ ID NO.: 1
GHRAAVNVVD
SEQ ID NO.: 2
RCIRFDNKRIVSGAY
SEQ ID NO.: 3
LVRCIRFDNKRIVSGAY
SEQ ID NO.: 4
AAVNVVDFDDKYIVSASG
SEQ ID NO.: 5
RAAVNVVDFDDKYIVSASG
SEQ ID NO.: 6
HRAAVNVVDFDDKYIVSASG
SEQ ID NO.: 7
HRAAVNVVDF
SEQ ID NO.: 8
AVNVVDFDDKYIVSASG
SEQ ID NO.: 9
TKHKWEAAHV
SEQ ID NO.: 10
GETRKVKAHSQT
SEQ ID NO.: 11
ETRKVKAHSQTHRVD
SEQ ID NO.: 12
GETRKVKAHSQTHRVD
SEQ ID NO.: 13
ETRKVKAHSQTHRVDLGT
SEQ ID NO.: 14
ETRKVKAHSQTHRVDLGTL
SEQ ID NO.: 15
GETRKVKAHSQTHRVDLGTL
SEQ ID NO.: 16
ETRKVKAHSQTHRVDLGTLRGY
SEQ ID NO.: 17
GETRKVKAHSQTHRVDLGTLRGY
SEQ ID NO.: 18
TKHKWEAAHVAEQ
SEQ ID NO.: 19
KWEAAHVAEQLRAY
SEQ ID NO.: 20
TKHKWEAAHVAEQLRAY
SEQ ID NO.: 21
TKHKWEAAHVAEQLRAYLEGT
SEQ ID NO.: 22
KWEAAHVAEQLRAYLEGTCVEW
SEQ ID NO.: 23
HEELVRCIRF
SEQ ID NO.: 24
EELVRCIRFD
SEQ ID NO.: 25
VRCIRFDNKRIVSGAY

In one or more further implementations, the Peptide Generator is configured to use a structure agnostic approach to generate a peptide having a relatively short sequence, less than 50 amino acids (AA), and a relatively high binding affinity for the proposed target. For example, the Peptide Generator is configured to provide the peptide of a peptide-E3 ubiquitin ligase fusion in which said peptide is one of the following of amino acid sequences:

SEQ ID No.: 28
DYEGSGSEAASLSSLNSSESDKDQ
SEQ ID No.: 29
YDSLLVFDYE
SEQ ID No.: 30
AADSDPTAPPYDSLLVFDYE
SEQ ID No.: 31
PTAPPYDSLLVFDYE
SEQ ID No.: 32
YDSLLVFDYEG
SEQ ID No.: 33
TAPPYDSLLVFDYE
SEQ ID No.: 34
DPTAPPYDSLLVFDYEGS
SEQ ID No.: 35
PTAPPYDSLLVFDYEG

In one or more further implementations, the Peptide Generator is configured to generate a peptide that has 60%, 65%, 70%, 75%, 80%, 85%, 90% homology with SEQ ID NOs. 1-25 and 28-35.

In yet a further implementation, any one of the derived peptide sequences are configured to bind to Beta-catenin. For example, any one of the derived peptide sequences are configured to bind to Beta-catenin having an amino acid sequence:

SEQ ID NO.: 26
MATQADLMELDMAMEPDRKAAVSHWQQQSYLDSGIHSGATTTAPSLSGKGNPEE
EDVDTSQVLYEWEQGFSQSFTQEQVADIDGQYAMTRAQRVRAAMFPETLDEGMQ
IPSTQFDAAHPTNVQRLAEPSQMLKHAVVNLINYQDDAELATRAIPELTKLLNDEDQ
VVVNKAAVMVHQLSKKEASRHAIMRSPQMVSAIVRTMQNTNDVETARCTAGTLHN
LSHHREGLLAIFKSGGIPALVKMLGSPVDSVLFYAITTLHNLLLHQEGAKMAVRLAG
GLQKMVALLNKTNVKFLAITTDCLQILAYGNQESKLIILASGGPQALVNIMRTYTYEKL
LWTTSRVLKVLSVCSSNKPAIVEAGGMQALGLHLTDPSQRLVQNCLWTLRNLSDA
ATKQEGMEGLLGTLVQLLGSDDINVVTCAAGILSNLTCNNYKNKMMVCQVGGIEAL
VRTVLRAGDREDITEPAICALRHLTSRHQEAEMAQNAVRLHYGLPVVVKLLHPPSH
WPLIKATVGLIRNLALCPANHAPLREQGAIPRLVQLLVRAHQDTQRRTSMGGTQQQ
FVEGVRMEEIVEGCTGALHILARDVHNRIVIRGLNTIPLFVQLLYSPIENIQRVAAGVL
CELAQDKEAAEAIEAEGATAPLTELLHSRNEGVATYAAAVLFRMSEDKPQDYKKRL
SVELTSSLFRTEPMAWNETADLGLDIGAQGEPLGYRQDDPSYRSFHSGGYGQDAL
GMDPMMEHEMGGHHPGADYPVDGLPDLGHAQDLMDGLPPGDSNQLAWFDTDL
CTNB1_HUMAN Catenin beta-1 OS = Homo sapiens OX = 9606
GN = CTNNB1 PE = 1 SV = 1.

In one or more further implementations, any one of the above derived peptides, or peptides having at least 70% homology therewith, are configured to bind to a Beta-catenin protein having an amino acid sequence that has 60%, 65%, 70%, 75%, 80%, 85%, 90% homology with SEQ ID NO.: 26.

In one or more further implementations, any one of the above derived peptides, or peptides having at least 70% homology therewith are linked to a degrader molecule, compound or complex. For example, the above derived peptides are linked to E3 ubiquitin ligase. In one or more implementations, E3 ubiquitin ligase is a eukaryotic E3 ubiquitin ligase. In a further implementation, the eukaryotic E3 ubiquitin ligase motif is a U-box motif. In a particular implementation, the eukaryotic E3 ligase motif is a human Carboxyl terminus of Hsc70-Interacting Protein (“CHIP (STUB1)”). In yet a further implementation, the human Carboxyl terminus of Hsc70-Interacting Protein (“CHIP (STUB1)”) has the TPR domain located at the CHIP(STUB1) N-terminus is deleted. In yet a further implementation, the E3 ubiquitin ligase has the amino acid sequence:

(SEQ ID No.: 27)
RLNFGDDIPSALRIAKKKRWNSIEERRIHQESELHSYLSRLIAAERERE
LEECQRNHEGDEDDSHVRAQQACIEAKHDKYMADMDELFSQVDEKRKKR
DIPDYLCGKISFELMREPCITPSGITYDRKDIEEHLQRVGHFDPVTRSP
LTQEQLIPNLAMKEVIDAFISENGWVEDY.

In one or more further implementations, the E3 ubiquitin ligase is an amino acid sequence that has 60%, 65%, 70%, 75%, 80%, 85%, 90% homology with SEQ ID NO.: 27.

While it is appreciated that the foregoing examples of a computationally derived guide or binder peptide or protein is linked to a E3 ubiquitin ligase, it will be understood by those possessing an ordinary level of skill in the requisite art that the E3 ligase motif is prokaryotic. Furthermore, in one or more implementations the E3 ligase motif is from a bacterial pathogen. For example, said bacterial pathogen is selected from the group consisting of Shigella, Salmonella, Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia.

Furthermore, the linked degrader described herein, can be selected from Shigella flexneri E3 ligase, SspH1, SspH2, SlrP, AvrPtoB, LubX, NleG5-1, NleG2-3, LegU1, LegAU13, NleL, SopA, SidC, XopL, GobX, VirF, GALA, AnkB, or SidE.

In a further implementation, the derived peptide and the E3 ligase are linked by a polypeptide linker of sufficient length to prevent the steric disruption of binding between the targeting domain and the biological target.

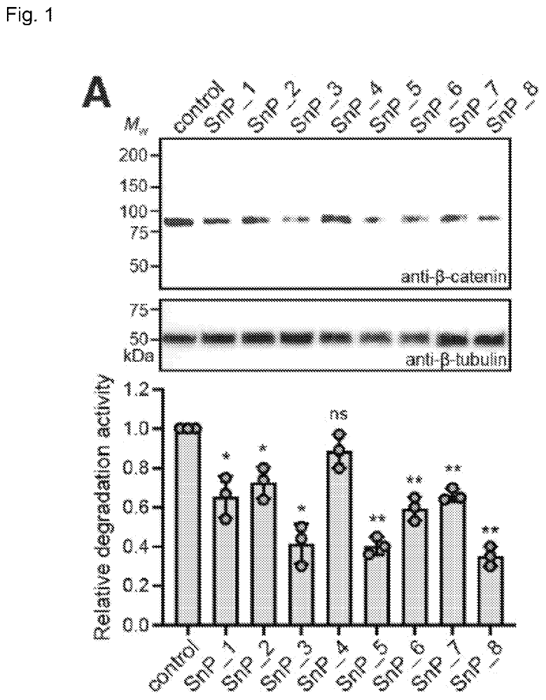

As further shown, and with particular reference to FIGS. 1-5, the inventors have found that the derived guide peptides and proteins, when incorporated into a uAb construct, are able to successfully provide for the degradation of endogenous β-catenin. More specifically, as shown herein, one or more computationally derived guide proteins (referred to herein individually as SnP_1 through SnP_8) are configured to provide the degradation of endogenous β-catenin. For example, and in no way limiting, the computational derived linkers derived using the peptide generation model described herein, when integrated into a uAb construct, are configured to provide the degradation of endogenous β-catenin in the cytosolic fraction of DLD1 cells.

As shown with respect to FIG. 1, the degradation of endogenous β-catenin was analyzed via immunoblotting and found to provide significant degradation of endogenous β-catenin relative to control cells. For ease of explanation, SEQ ID Nos. 28-35, referred to as SnP_1-SnP_8 respectively, were used to evaluate if the derived uAbs could degrade endogenous, cytosolic β-catenin. Here, each of SnP_1-SnP_8 were transiently transfected with pCMV plasmid encoding each of the candidate uAbs. It will be understood that the control lane was non-transfected DLD1 cells. An equivalent amount of total protein was loaded in each lane. Blots were probed with anti-β-catenin and anti-β-tubulin antibodies and are representative of three biological replicates. Relative degradation activity was determined by densitometry analysis of anti-β-catenin immunoblot using ImageJ software. Data represent the mean±SD from three independent experiments. Statistical significance was determined by Welch's two-sided t-test (*p<0.05; **p<0.01; ns, not significant).

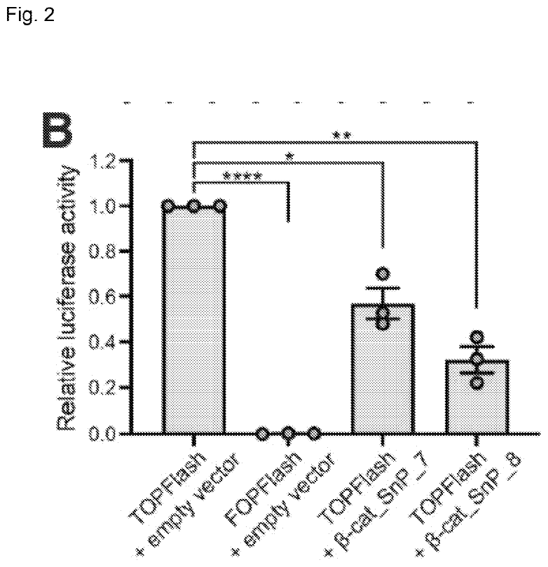

As shown in FIG. 2, a TOPFlash reporter plasmid and CMV-Renilla reporter plasmid were co-transfected into DLD1 cells with pcDNA3-β-cat_SnP_7 uAb, pcDNA3-β-cat_SnP_8 uAb, or empty pcDNA3 vector. Luciferase activities were measured and normalized against the control Renilla activities. The luciferase activity of DLD1 cells transfected with an empty vector was arbitrarily set to 1. The FOPFlash reporter has multiple copies of mutated TCF binding sites and served as a negative control. Data represent the mean±SD from three independent experiments. Statistical significance was determined by Welch's two-sided t-test (*, p value=0.023; **, p value=0.007; ****, p value<0.0001).

As shown in FIG. 2, co-transfection of DLD1 cells with the TOPFLash reporter plasmid along with the strong SnP_8 degrader was observed to dramatically decrease the transcriptional response to β-catenin from the TOPFlash reporter compared to empty vector control cells. For comparison, the less potent SnP_7 degrader was observed to induce a more modest inhibitory effect on β-catenin signaling, in line with its intermediate degradation activity.

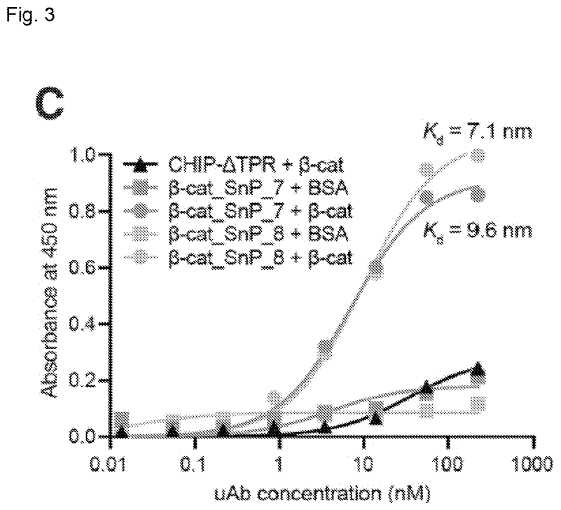

Turning now to FIG. 3, the β-catenin binding activity for uAbs derived using the peptide model, was determined by ELISA. Purified β-cat_SnP_7 and β-cat_SnP_8 uAbs were assayed for binding to immobilized β-catenin or a bovine serum albumin (BSA) control protein. Purified CHIPΔTPR lacking a substrate-binding domain served as a negative control. Data represent the mean±SD from three independent experiments, with error bars smaller than the data points. Values of the equilibrium dissociation constant (Kd) were determined by non-linear regression analysis in GraphPad Prism 9 software. Luciferase activities were measured and normalized against the control Renilla activities. The luciferase activity of DLD1 cells transfected with an empty vector was arbitrarily set to 1. The FOPFlash reporter has multiple copies of mutated TCF binding sites and served as a negative control. Data represent the mean¬±SD from three independent experiments. Statistical significance was determined by Welch's two-sided t-test (*, p value=0.023; **, p value=0.007; ****, p value<0.0001).

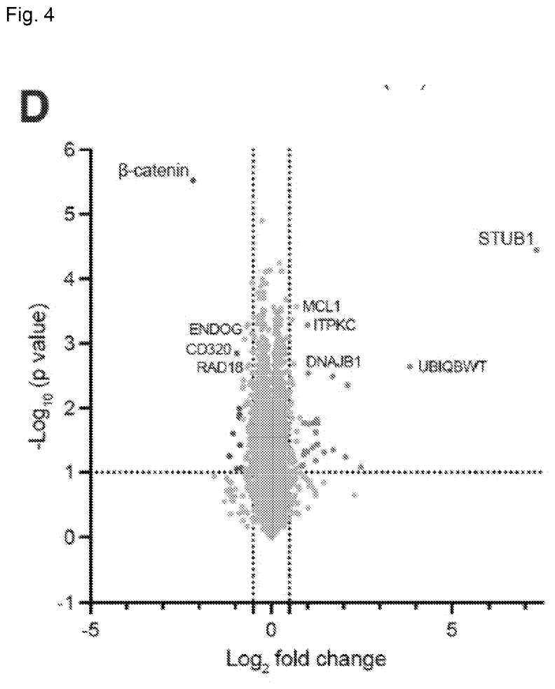

As shown in FIG. 4, a volcano plot of differentially abundant proteins was generated. Here, HEK293T cells were transfected with pcDNA3-β-cat_SnP_8 uAb (shown) alongside a β-catenin-sfGFP expressing vector, and total protein was collected for nano LC-MS/MS. Data were log2-normalized and fold-change and p-value (unpaired, two-tailed t-test) was performed in Excel and plotted in GraphPad Prism 9 software. It will be understood that in this particular context, STUB1 refers to the quantities of overexpressed CHIP E3 ligase.

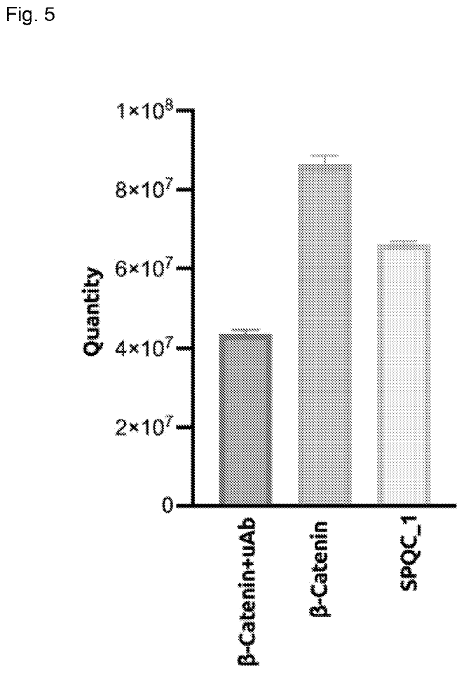

As shown in the FIGS. 4 and 5, there was the expected increase in uAb-associated proteins, including tryptic peptides assigned to the human CHIP protein (STUB1), and a corresponding decrease in β-catenin abundance between the control and treated samples for both tested uAbs. In contrast, there were no significant changes in the abundance of other proteins as a function of uAb expression, confirming that there were no statistically significant off-target effects associated with uAb expression or degradation.

Thus, the demonstrated approach can be used to generate guide peptides that when incorporated into a uAb provide for degradation of the target in vitro. The resulting uAbs exhibit robust degradation capabilities, high nanomolar binding affinities, low off-targeting propensities, and can affect downstream signaling pathways post-target degradation.

It will be further appreciated that other guide or binder proteins can be used to degrade β-catenin. For example, in addition to computationally-designed peptide binders, genetically-encoded binders such as antibody/nanobody and non-antibody scaffolds. These antibody/nanobody and non-antibody scaffolds can be linked to an E3 ubiquitin ligase or other described degraders to provide for targeted degradation of the intended biological target.

For example, the targeting domain of a uAb can include but is not limited to any of the foregoing antibodies/nanobodies: antibody, polyclonal antibody, monoclonal antibody, recombinant antibody, antibody fragment, Fab′, F(ab′)2, Fv, scFv, tascFvs, bis-scFvs, sdAb, VH, VL, VLR, Vnar, scAb, humanized antibody, chimeric antibody, complementary determining region (CDR), nanobody, intrabody, unibody, minibody, and VHH.

Likewise, non-antibody scaffolds can be used to form a uAb for targeted degradation by linked E3 ubiquitin ligases. Such non-antibody scaffolds include but are not limited to Adnectins, Affibodies, Affilins, Anticalins, Atrimers, Bicyclic peptides, Centyrins, Cys-knots, DARPins, Fynomers, Kunitz domains, Obodies, Pronectins, Fn3s, Knottins, and Sso7d.

Such antibody/nanobody and non-antibody scaffolds can, in one or more implementations, be developed by either experimental screening or computational design methodologies. These approaches can allow the E3 ubiquitin ligases to be linked to the selected antibody/nanobody and non-antibody scaffold and achieve targeted degradation of the intended biological target.

### Methods of Treatment

In one or more implementations, a therapeutic is provided where the therapeutic includes the polynucleotide that includes of any of sequences SEQ ID NO.: 1-24 and 28-35, or a sequence having 80% homology thereto.

In one or more implementations, a method is provided to inhibit the function of β-catenin in tumorigenesis. In one particular implementation, the method includes selectively blocking the cytosolic/nuclear activity of hypophosphorylated β-catenin while leaving the membrane activity of β-catenin intact. The method further includes administering to a patient in need thereof, an amount of peptide-E3 ligase fusion sufficient to selectively block the cytosolic/nuclear activity of hypophosphorylated β-catenin while leaving the membrane activity of β-catenin intact.

In one further implementation, the peptide therapeutic includes any of the foregoing polynucleotides coupled a delivery vector in which said delivery vector may be either a virus or micelle. In a further arrangement, the described peptide-based therapeutic includes the fusions of any of the foregoing polynucleotides in which said peptide fusion is further fused to a cell penetrating motif or a cell surface receptor binding motif. In certain embodiments, the compositions and methods of the present disclosure are useful for the prevention and/or treatment of symptoms of cancer and metastasis.

In one embodiment, the subject has a cancer and metastasis. In some embodiments, the cancer or metastasis is selected from the group of basal cell carcinoma (BCC), head and neck squamous cell carcinoma (HNSCC), prostate cancer (CaP), pilomatrixoma (PTR) and medulloblastoma (MDB).

In one or more implementations the following particular embodiments are understood and appreciated:

Implementation 1: A method of generating a peptide-E3 ubiquitin ligase fusion comprising the steps of:
a. Identifying a biological target for E3 ubiquitin degradation,
b. Providing a nucleotide sequence that corresponds to the biological target to a peptide generation module, configured as code executing in a computer environment, wherein the peptide generation module is configured to generate a target nucleotide sequence for a peptide that binds to the biological target,
c. Generating a peptide-E3 ubiquitin ligase fusion incorporating the target peptide, a linker and E3 ubiquitin ligase; and
d. Synthesizing the peptide-E3 ubiquitin ligase fusion.

Implementation 2: The method of any previous implementation, wherein the target is Beta-Catenin.

Implementation 3 The method of any previous implementation, wherein the synthesized peptide-E3 ubiquitin ligase fusion inhibits the function of beta-catenin in tumorigenesis when administered to a patient.

Implementation 4: The method of any previous implementation wherein the generated peptide has a sequence ID corresponding to one of SEQ ID Nos. 1-25 and SEQ ID Nos.: 28-35.

Implementation 5: An isolated chimeric molecule comprising: (i) a degradation domain comprising an E3 ubiquitin ligase motif without lysine residues; (ii) a targeting domain comprising a Beta-Catenin binding motif which is heterologous to the E3 ubiquitin ligase motif; and (iii) a linker coupling said degradation domain to said targeting domain.

Implementation 6: The isolated chimeric molecule of any previous implementation wherein where the length of the targeting domain is less than 50 amino acids.

Implementation 7: The isolated chimeric molecule of any previous implementation wherein the equilibrium dissociation constant of the targeting domain for Beta-Catenin is at least 5.0.

Implementation 8: The isolated chimeric molecule of any previous implementation wherein the targeting domain peptide has a sequence of any of the amino acid sequence SEQ ID No.: 1-SEQ ID NO.: 24 and SEQ ID No. 28-35.

Implementation 9: The isolated chimeric molecule of any previous implementation, wherein the targeting domain peptide has a sequence of one of: SEQ ID Nos. 28-35.

Implementation 10: The isolated chimeric molecule of any previous implementation, wherein the targeting domain peptide is an amino acid sequence possessing sequence homology of greater than 80% to any of the amino acid sequences SEQ ID No.: 1-SEQ ID NO.: 24 and SEQ ID No.: 28-35.

Implementation 11: The chimeric molecule of any previous implementation, wherein said linker is a polypeptide linker of sufficient length to prevent the steric disruption of binding between said targeting domain and said protein substrate.

Implementation 12: The isolated chimeric molecule of any previous implementation wherein the isolated chimeric molecules is coupled to delivery vector in which the delivery vector is a lipid nano particle or adeno-associated vectors.

Implementation 13: The isolated chimeric molecule of any previous implementation wherein the targeting domain binds to Beta-Catenin having an amino acid sequence of SEQ ID No.: 26.

Implementation 14: The isolated chimeric molecule of any previous implementation wherein the E3 ubiquitin ligase has an amino acid sequence of SEQ ID No. 27.

Implementation 15: The isolated chimeric molecule of any previous implementation wherein the E3 ubiquitin ligase motif is a human Carboxyl terminus of Hsc70-Interacting Protein (“CHIP (STUB1)”) whose TPR domain located at the CHIP (STUB1) N-terminus is deleted.

Implementation 16: The method of any previous implementation, wherein the derived peptide is configured to bind to an E3 ubiquitin ligase of amino acid sequence:

RLNFGDDIPSALRIAKKKRWNSIEERRIHQESELHSYLSRLIAAERERE
LEECQRNHEGDEDDSHVRAQQACIEAKHDKYMADMDELFSQVDEKRKKR
DIPDYLCGKISFELMREPCITPSGITYDRKDIEEHLQRVGHFDPVTRSP
LTQEQLIPNLAMKEVIDAFISENGWVEDY.

Implementation 17: A method of treating a cancer comprising: administering the isolated chimeric molecule of any previous implementation to a patient suffering from a cancer, wherein a caner state is marked by the presence of endogenous, cytosolic β-catenin.

Implementation: 18 The method of any previous implementation, wherein the isolated chimeric molecule is coupled a delivery vector in which said delivery vector may be either a virus or micelle.

Implementation 19: The method of any previous implementation, wherein the isolated chimeric molecule is further fused to a cell penetrating motif or a cell surface receptor binding motif.

Implementation 20: The method of any previous implementation, wherein the isolated chimeric molecule is coupled a delivery vector in which said delivery vector is a lipid nano particle.

### Pharmaceutical Compositions

The present disclosure thus provides pharmaceutical compositions that include Peptide-E3 ubiquitin ligase fusion compounds and a pharmaceutically acceptable carrier. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein. It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the Peptide-E3 ubiquitin ligase fusion based treatments may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof.

Suitable lubricants include, but are not limited to, sodium stearyl fumarate, stearic acid, polyethylene glycol or stearates, such as magnesium stearate.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide.

Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The Peptide-E3 ubiquitin ligase fusion based treatments of the present disclosure may also be administered via infusion or injection (e.g., using needle (including microneedle) injectors and/or needle-free injectors). Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject.

A Peptide-E3 ubiquitin ligase fusion based treatment described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

A Peptide-E3 ubiquitin ligase fusion based treatment described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5 mg to about 5 mg, from about 0.5 mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the Peptide-E3 ubiquitin ligase fusion based treatment is administered for a period of at least one day. In other embodiments, the Peptide-E3 ubiquitin ligase fusion based treatment is administered for a period of at least 2 days. In other embodiments, the Peptide-E3 ubiquitin ligase fusion based treatment is administered for a period of at least 3 days. In other embodiments, the Peptide-E3 ubiquitin ligase fusion based treatment is administered for a period of at least 4 days. In other embodiments, the Peptide-E3 ubiquitin ligase fusion based treatment is administered for a period of at least 5 days. In other embodiments, the Peptide-E3 ubiquitin ligase fusion based treatment is administered for a period of at least 6 days. In other embodiments, the Peptide-E3 ubiquitin ligase fusion based treatment is administered for a period of at least 7 days. In other embodiments, the Peptide-E3 ubiquitin ligase fusion based treatment is administered for a period of at least 10 days. In other embodiments, the Peptide-E3 ubiquitin ligase fusion based treatment is administered for a period of at least 14 days. In other embodiments, the Peptide-E3 ubiquitin ligase fusion based treatment is administered for a period of at least one month. In some embodiments, the Peptide-E3 ubiquitin ligase fusion based treatment is administered chronically for as long as the treatment is needed.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

What is claimed is:

1. A method of generating a peptide-E3 ubiquitin ligase fusion comprising the steps of:
1. Identifying a biological target for E3 ubiquitin degradation,
2. Providing a nucleotide sequence that corresponds to the biological target to a peptide generation module, configured as code executing in a computer environment, wherein the peptide generation module is configured to generate a target nucleotide sequence for a peptide that binds to the biological target,
3. Generating a peptide-E3 ubiquitin ligase fusion incorporating the target peptide, a linker and E3 ubiquitin ligase; and
4. Synthesizing the peptide-E3 ubiquitin ligase fusion.

2. The method of claim 1, wherein the target is Beta-Catenin.

3. The method of claim 1, wherein the synthesized peptide-E3 ubiquitin ligase fusion inhibits the function of beta-catenin in tumorigenesis when administered to a patient.

4. The method of claim 1 wherein the generated peptide has a sequence ID corresponding to one of SEQ ID Nos. 1-25 and SEQ ID Nos.: 28-35.

5. An isolated chimeric molecule comprising: (i) a degradation domain comprising an E3 ubiquitin ligase motif without lysine residues; (ii) a targeting domain comprising a Beta-Catenin binding motif which is heterologous to the E3 ubiquitin ligase motif; and (iii) a linker coupling said degradation domain to said targeting domain.

6. The isolated chimeric molecule of claim 5 wherein where the length of the targeting domain is less than 50 amino acids.

7. The isolated chimeric molecule of claim 6 wherein the equilibrium dissociation constant of the targeting domain for Beta-Catenin is at least 5.0.

8. The isolated chimeric molecule of claim 5 wherein the targeting domain peptide has a sequence of any of the amino acid sequence SEQ ID No.: 1-SEQ ID NO.: 24 and SEQ ID No. 28-35.

9. The isolated chimeric molecule of claim 8, wherein the targeting domain peptide has a sequence of one of: SEQ ID Nos. 28-35.

10. The isolated chimeric molecule of claim 5, wherein the targeting domain peptide is an amino acid sequence possessing sequence homology of greater than 80% to any of the amino acid sequences SEQ ID No.: 1-SEQ ID NO.: 24 and SEQ ID No.: 28-35.

11. The chimeric molecule of claim 5, wherein said linker is a polypeptide linker of sufficient length to prevent the steric disruption of binding between said targeting domain and said protein substrate.

12. The isolated chimeric molecule of claim 5 wherein the isolated chimeric molecules is coupled to delivery vector in which the delivery vector is a lipid nano particle or adeno-associated vectors.

13. The isolated chimeric molecule of claim 5 wherein the targeting domain binds to Beta-Catenin having an amino acid sequence of SEQ ID No.: 26.

14. The isolated chimeric molecule of claim 5 wherein the E3 ubiquitin ligase has an amino acid sequence of SEQ ID No. 27.

15. The isolated chimeric molecule of claim 5 wherein the E3 ubiquitin ligase motif is a human Carboxyl terminus of Hsc70-Interacting Protein (“CHIP (STUB1)”) whose TPR domain located at the CHIP(STUB1) N-terminus is deleted.

16. The method of claim 1, wherein the derived peptide is configured to bind to an E3 ubiquitin ligase of amino acid sequence: RLNFGDDIPSALRIAKKKRWNSIEERRIHQESELHSYLSRLIAAERERELEEC QRNHEGDEDDSHVRAQQACIEAKHDKYMADMDELFSQVDEKRKKRDIPDY LCGKISFELMREPCITPSGITYDRKDIEEHLQRVGHFDPVTRSPLTQEQLIPNL AMKEVIDAFISENGWVEDY.

17. A method of treating a cancer comprising:
administering the isolated chimeric molecule of claim 5 to a patient suffering from a cancer, wherein a caner state is marked by the presence of endogenous, cytosolic β-catenin.

18. The method of claim 17, wherein the isolated chimeric molecule is coupled a delivery vector in which said delivery vector may be either a virus or micelle.

19. The method of claim 17, wherein the isolated chimeric molecule is further fused to a cell penetrating motif or a cell surface receptor binding motif.

20. The method of claim 17, wherein the isolated chimeric molecule is coupled a delivery vector in which said delivery vector is a lipid nano particle.


SEQUENCE LISTING (27 sequences)

SEQ ID NO: 1 - protein, 10 aa, synthetic construct
GHRAAVNVVD

SEQ ID NO: 2 - protein, 15 aa, synthetic construct
RCIRFDNKRIVSGAY

SEQ ID NO: 3 - protein, 17 aa, synthetic construct
LVRCIRFDNKRIVSGAY

SEQ ID NO: 4 - protein, 18 aa, synthetic construct
AAVNVVDFDDKYIVSASG

SEQ ID NO: 5 - protein, 19 aa, synthetic construct
RAAVNVVDFDDKYIVSASG

SEQ ID NO: 6 - protein, 20 aa, synthetic construct
HRAAVNVVDFDDKYIVSASG

SEQ ID NO: 7 - protein, 10 aa, synthetic construct
HRAAVNVVDF

SEQ ID NO: 8 - protein, 17 aa, synthetic construct
AVNVVDFDDKYIVSASG

SEQ ID NO: 9 - protein, 10 aa, synthetic construct
TKHKWEAAHV

SEQ ID NO: 10 - protein, 12 aa, synthetic construct
GETRKVKAHSQT

SEQ ID NO: 11 - protein, 15 aa, synthetic construct
ETRKVKAHSQTHRVD

SEQ ID NO: 12 - protein, 16 aa, synthetic construct
GETRKVKAHSQTHRVD

SEQ ID NO: 13 - protein, 18 aa, synthetic construct
ETRKVKAHSQTHRVDLGT

SEQ ID NO: 14 - protein, 19 aa, synthetic construct
ETRKVKAHSQTHRVDLGTL

SEQ ID NO: 15 - protein, 20 aa, synthetic construct
GETRKVKAHSQTHRVDLGTL

SEQ ID NO: 16 - protein, 22 aa, synthetic construct
ETRKVKAHSQTHRVDLGTLRGY

SEQ ID NO: 17 - protein, 23 aa, synthetic construct
GETRKVKAHSQTHRVDLGTLRGY

SEQ ID NO: 18 - protein, 13 aa, synthetic construct
TKHKWEAAHVAEQ

SEQ ID NO: 19 - protein, 14 aa, synthetic construct
KWEAAHVAEQLRAY

SEQ ID NO: 20 - protein, 17 aa, synthetic construct
TKHKWEAAHVAEQLRAY

SEQ ID NO: 21 - protein, 21 aa, synthetic construct
TKHKWEAAHVAEQLRAYLEGT

SEQ ID NO: 22 - protein, 22 aa, synthetic construct
KWEAAHVAEQLRAYLEGTCVEW

SEQ ID NO: 23 - protein, 10 aa, synthetic construct
HEELVRCIRF

SEQ ID NO: 24 - protein, 10 aa, synthetic construct
EELVRCIRFD

SEQ ID NO: 25 - protein, 16 aa, synthetic construct
VRCIRFDNKRIVSGAY

SEQ ID NO: 26 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 27 - protein, 176 aa, synthetic construct
RLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY